(19) United States
(12) Patent Application Publication
(10) Pub. No.: US 2008/0207915 A1
(43) Pub. Date: Aug. 28, 2008

(54) Process for the Preparation of 2,2'-[5-(1H-1,2,4-Triazole-1-Ylmethyl) -1,3-Phenylene] Di (2-Methylpropionitrile)

(76) Inventors: Tarur Venkatasubramanian Radhakrishnan, Maharashtra (IN); Bhise Nandu Baban, Maharashtra (IN); Sathe Dhananjay Govind, Maharashtra (IN); Chaudhari Chhayendra Janardan, Maharashtra (IN); Joshi Mehul Ashokkumar, Maharashtra (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 12/088,950

(22) PCT Filed: Oct. 5, 2005
(86) PCT No.: PCT/IN2005/000332

§ 371 Date: Apr. 2, 2008

(51) Int. Cl. C07D 249/08; C07C 22/04

(52) U.S. Cl. 548/267.4; 570/190

(57) ABSTRACT

The present invention discloses a process for the preparation of Anastrozole of the formula I in high purity and in high yield. 3,5-bis(halomethyl)toluene is prepared by reacting mesitylene with N-halosuccinimide in the presence of light or dibenzoyl peroxide or azobis isobutyronitrile as a catalyst and in a chlorinated solvent. 3,5-bis(halomethyl)toluene is cyanated with metal cyanide in the presence of a catalyst and in water, organic solvent or mixture thereof at temperature of 40 to 60° C. to obtain 2,2′-(5-methyl-1,3 phenylene)diacetonitrile which is further methylated with iodomethane in the presence of base and an organic solvent at temperature of 0 to 15° C. to obtain 2,2′-(5-methyl-1,3-phenylene)di(2-methyl-propiononitrile). The product obtained is treated with N-halosuccinimide in the presence of a catalyst and in a chlorinated solvent at temperature of 60 to 100° C. to obtain 2,2′-(5-halomethyl-1,3-phenylene)di(2-methyl propionitrile) which was further treated with potassium or sodium salt 1,2,4-triazole at temperature of 20 to 50° C. in dimethyl formamide to obtain crude 2,2′-[5-(1H-1,2,4-triazole-1-ylmethyl)-1,3-phenylene]di(2-methyl-propionitrile). The crude product is purified by column chromatography using a stationary phase and a mobile phase followed by recrystallization with a solvent or mixture of solvents to obtain highly pure Anastrozole.

### TECHNICAL FIELD

The invention relates to a process for the preparation of 2,2′-[5-(1H-1,2,4-triazole-1-ylmethyl)-1,3-phenylene]di(2-methylpropionitrile) of the formula (I) in high yield and in high purity.

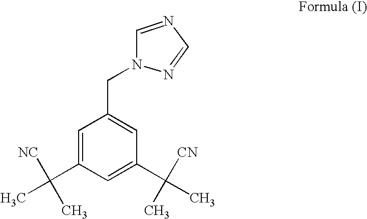

The invention also relates to highly pure 2,2′-[5-(1H-1,2,4-triazole-1-ylmethyl)-1,3-phenylene]di (2-methylpropionitrile) of the formula (I) in high yield prepared by the above process.

2,2′-[5-(1H-1,2,4-triazole-1-ylmethyl)-1,3-phenylene]di(2-methylpropionitrile) is also known as α,α,α′,α′-tetramethyl-5-(1H-1,2,4-triazole-1-ylmethyl)-1,3-benzene diacetonitrile and has the generic name Anastrozole. It is a potent and selective non-steroidal aromatase inhibitor. Aromatase inhibitors are a class of compounds that act systematically to inhibit oestrogen synthesis in tissues and prevent oestrogen biosynthesis by inhibiting the enzyme aromatase, which catalyses the conversion of adrenal androgens (androstenedione and testosterone) to oestrogens (oestrogen and oestradiol). Anastrozole is used for hormone responsive breast cancer in post-menopausal women as it significantly lowers serum estradiol concentrations and has no detectable effect on formation of adrenal corticosteroids or aldosterone.

### BACKGROUND OF THE INVENTION

Anastrozole was first disclosed in U.S. Pat. No. 4,935,437, which was reissued as US RE 36,617. This patent describes two routes for the preparation of Anastrozole. First route comprises bromination of 2,2′-(5-methyl-1,3-phenylene)di(2-methyl propionitrile) of the formula (II)

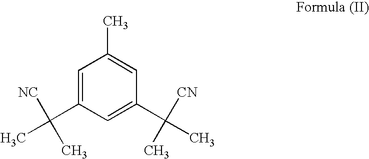

with N-bromosuccinimide in the presence of the catalyst, benzoyl peroxide, and carbon tetrachloride as the solvent at reflux temperature followed by evaporation of the solvent from the reaction mixture to dryness to obtain a residue comprising 2,2′-(5-bromomethyl-1,3-phenylene)di(2-methylpropionitrile) of the formula (IIIb).

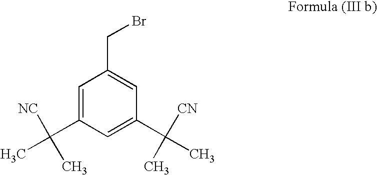

The residue was dissolved in dimethyl formamide and treated with sodium triazole followed by dilution with water and extraction with ethyl acetate. The extract was evaporated to dryness under reduced pressure to give crude Anastrozole which was purified by flash column chromatography with ethyl acetate as eluent and crystallized with ethyl acetate/cyclohexane.

The compound of the formula (II) is prepared from 3,5-bis(bromomethyl)toluene of the formula (Vb)

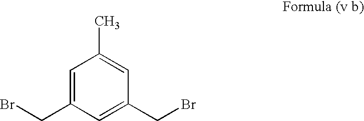

by treating it with potassium cyanide in the presence of tetrabutyl ammonium bromide as the catalyst and dichloromethane and water as the solvent at reflux temperature followed by extraction with ethyl acetate and drying to get 2,2′-(5-methyl-1,3-phenylene)diacetonmtrile of the formula (VI).

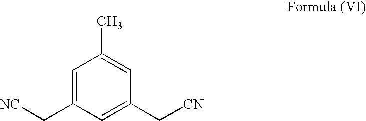

The compound of formula (VI) is purified by flash column chromatography using petroleum ether and ethyl acetate as eluent followed by crystallization with carbon tetrachloride. The compound of the formula (VI) is treated with iodomethane in presence of the base such as sodium hydride and dimethyl formamide followed by extraction with ethyl acetate and evaporation to dryness under reduced pressure to obtain 2,2′-(5-methyl-1,3-phenylene)di(2-methyl propionitrile) of the formula (II)

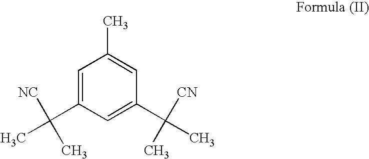

The compound of formula (I) is purified by crystallization using carbon tetrachloride.

Preparation of 3,5-bis(bromomethyl) toluene of the formula (V b) from mesitylene using carbon tetrachloride is disclosed in the prior art (Mitchell R. H. et al, J. Chem., 52 (1974), 3054-3056).

As all the intermediate compounds are purified either by using flash chromatography and crystallization or by crystallization, the above route is tedious and time consuming to be carried out. It requires large volumes of the solvents and is not economical. Further the preparation of 2,2′-(5-bromomethyl-1,3-phenylene)di(2-methylpropionitrile) of the formula (IIIb) comprises carbon tetrachloride as a solvent which is known for ozone depletion and is not environment friendly. The US patent does not disclose yield and purity of the product, Anastrozole, obtained by the above process but when we carried out the procedure of the above route in our laboratory, the yield and purity of Anastrozole obtained were found to be about 3 to 5% and about 99.5%, respectively.

The second route comprises reacting 2,2′-(5-chloromethyl-1,3-phenylene)di(2-methyl propionitrile) of the formula (IIIa)

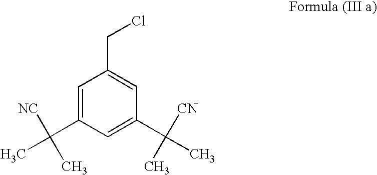

with 1H-1,2,4-triazole in acetonitrile at reflux temperature followed by evaporation of the solvent to obtain a residue which is partitioned between 1N aqueous potassium hydrogen carbonate solution and ethyl acetate. The organic phase is separated and dried under reduced pressure to get a residue comprising a mixture of Anastrozole and its isomer, 2,2′-[5-(4H-1,2,4-triazol-4-ylmethyl)-1,3-phenylene]di(2-methyl propionitrile) of the formula (IV).

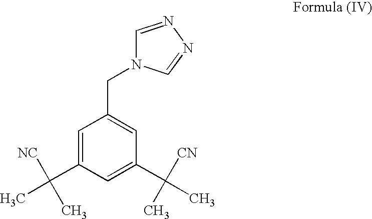

The residue is purified by flash column chromatography using methanol and chloroform as eluent to obtain Anastrozole of the formula (I). Further elution with methanol and chloroform gave the compound of the formula (IV).

The starting material of the formula (IIIa) is prepared from 3,5-dimethylbenzoate of the formula (VII)

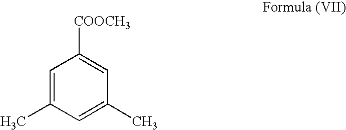

by treating it with N-bromosuccinimide in the presence of benzoyl peroxide as the catalyst and carbon tetrachloride as the solvent at reflux temperature. The reaction mixture is filtered and the filtrate is evaporated to dryness under reduced pressure to give a residue which is crystallized with cyclohexane to give methyl-3,5-bis(bromomethyl)benzoate of the formula (VIII).

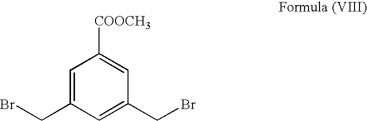

The compound of the formula (VIE) is treated with potassium cyanide in the presence of tetrabutyl ammonium bromide as the catalyst and dichloromethane and water as the solvent at reflux temperature followed by extraction with ethyl acetate. The extract is dried to get methyl 3,5-bis(cyano methyl)benzoate of the formula (IX)

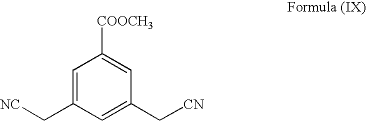

The compound of the formula (IX) is purified by flash column chromatography with petroleum ether and ethyl acetate as eluent followed by crystallization with carbon tetrachloride and allylated with methyl iodide in the presence of base sodium hydride as the catalyst in dimethyl formamide to obtain methyl 3,5-bis(1-cyano-1-methyl ethyl)benzoate of the formula (X).

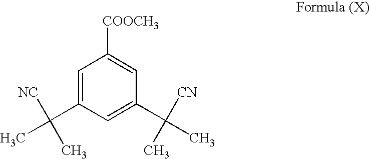

The compound of the formula (X) is reduced with lithium borohydride in tetrahydrofuran at reflux temperature. The reaction mixture is acidified with aqueous hydrochloric acid and extracted with ethyl acetate. The extract is washed with potassium bicarbonate and evaporated to dryness under reduced pressure to obtain 2,2′-(5-hydroxymethyl-1,3-phenylene)di(2-methylpropionitrile) of the formula (XI).

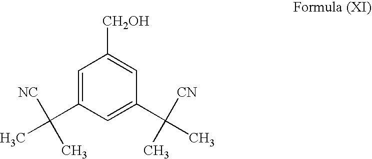

The compound of the formula (XI) is chlorinated with thionyl chloride in the presence of pyridine and dichloromethane followed by evaporation of the reaction mixture to dryness under reduced pressure. The residue is partitioned between ethyl acetate and water and the organic phase is evaporated to dryness under reduced pressure to obtain the compound of formula (IIIa).

The second route uses lithium borohydride, which is moisture sensitive and pyrophoric and difficult to handle at industrial scale. This route also uses carbon tetrachloride solvent, which is ozone depleting and environmentally harmful. It gives mixture of Anastrozole and its isomer, namely compound of formula (IV).

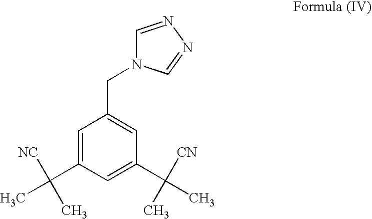

The isomeric mixture is separated by flash column chromatography and purified by crystallization. The intermediate compounds are purified by flash chromatography or extraction or crystallization or combination thereof. Therefore, the second route is also tedious and time consuming to be carried out, requires large volumes of solvents and is uneconomical.

### OBJECTS OF THE INVENTION

An object of the invention is to provide a process for the preparation of 2,2′-[5-(1H-1,2,4-triazole-1-ylmethyl)-1,3-phenylene]di(2-methylpropionitrile) of the formula (I) in high yield and in high purity.

Another object of the invention is to provide a cost effective, efficient, economical and environment friendly process for preparation of 2,2′-[5-(1H-1,2,4-triazole-1-ylmethyl)-1,3-phenylene]di(2-methylpropionitrile) of the formula (I).

Yet another object of the invention is to provide a process for the preparation of 2,2′-[5-(1H-1,2,4-triazole-1-ylmethyl)-1,3-phenylene]di(2-methylpropionitrile) of the formula (I), which reduces the process duration.

Yet another object of the invention is to provide highly pure 2,2′-[5-(1H-1,2,4-triazole-1-ylmethyl)-1,3-phenylene]di(2-methylpropionitrile) of the formula (I) in high yield as obtained by the above process.

Yet another object of the invention is to provide a process for the preparation of 3,5-bis(halomethyl)toluene of the formula (V) from mesitylene.

### DETAILED DESCRIPTION OF THE INVENTION

According to the invention there is provided a process for the preparation of 2,2′-[5-(1H-1,2,4-triazole-1-ylmethyl)-1,3-phenylene]di(2-methylpropionitrile) of the formula (I)

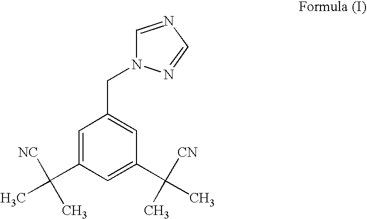

comprising the following steps
a. preparing 3,5-bis(halomethyl)toluene of the formula (V)

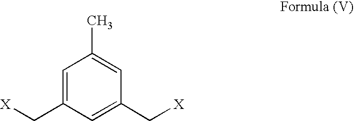

wherein X is Cl (a); Br (b) or I (c)
by reacting mesitylene with N-halosuccinimide in a chlorinated solvent selected from dichloroethane, 1,1,1-trichloroethane, 1,1,2-ethylenetrichloride, tetrachloroethane, dichloromethane or chlorobenzene or mixtures thereof in the presence of a catalyst selected from visible light or dibenzoyl peroxide or azobis isobutyronitrile at a temperature of 60 to 100° C.;
b. preparing 2,2′-(5-methyl-1,3-phenylene)diacetonitrile of the formula (VI)

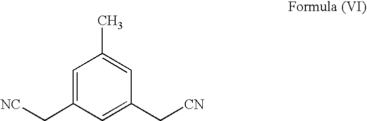

by cyanation of the compound of the formula (V) with metal cyanide in water or organic solvent or mixture thereof in the presence of a catalyst at a temperature of 40 to 60° C.;
c. preparing 2,2′-(5-methyl-1,3-phenylene)di(2-methyl-propiononitrile) of the formula (II)

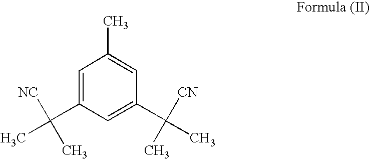

by methylating the compound of the formula (VI) with iodomethane in the presence of base and an organic solvent at temperature of 0 to 15° C.;
d. preparing 2,2′-(5-halomethyl-1,3-phenylene)di(2-methyl propionitrile) of the formula (I)

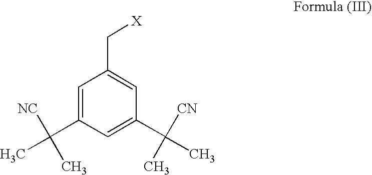

wherein X is Cl (a) or Br (b) or I (c)
by halogenating the compound of the formula (II) with N-halosuccinimide in a chlorinated solvent selected from ethylene dichloride, 1,1,1-trichloroethane, 1,1,2-ethylene trichloride or tetrachloroethane in the presence of a catalyst at temperature of 60 to 100° C.;
e. preparing 2,2′-[5-(1H-1,2,4-triazole-1-ylmethyl)-1,3-phenylene]di(2-methylpropionitrile) of the formula (I) by treating the compound of the formula (III) with potassium or sodium salt of 1,2,4-triazole in dimethyl formamide at temperature of 20 to 50° C.; and
f. purifying the compound of the formula (I) by column chromatography using a stationary phase and a mobile phase followed by recrystallization with a solvent or mixture of solvents to obtain highly pure compound of the formula (I).

According to the present invention there is also provided a process for the preparation of 3,5-bis(halomethyl)toluene of the formula (V)

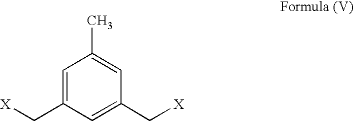

wherein X is Cl (a); Br (b) or I (c)
   comprising reacting mesitylene with N-halosuccinimide in a chlorinated solvent selected from dichloroethane, 1,1,1-trichloroethane, 1,1,2-ethylenetrichloride, tetrachloroethane, dichloromethane or chlorobenzene or mixtures thereof in the presence of a catalyst selected from visible light or dibenzoyl peroxide or azobis isobutyronitrile at a temperature of 60 to 100° C.

The N-halosuccinimide used in the reaction of mesitylene is N-bromosuccinimide, N-chlorosuccinimide or N-iodosuccinimide or preferably is N-bromosuccinimide. Preferably the chlorinated solvent used in the reaction of mesitylene is 1,1,2-ethylenetrichloride. Preferably the catalyst used in the reaction of mesitylene is visible light. Preferably the reaction of mesitylene is carried out at 88 to 92° C.

The compound of the formula (V) is further purified by recystallization using a solvent selected from alcohols such as ethanol, methanol, n-propanol, isopropanol, etc; ketones such as acetone, methylisobutyl ketone, methylethylketone, etc.; ethers; esters; aliphatic or aromatic hydrocarbon solvents such as hexane, toluene, xylene or halogenated solvents such methylene chloride, etc or mixtures thereof or preferably is mixture of toluene and isopropanol.

According to the invention there is provided a 3,5-bis(halo methyl)toluene obtained by the above process. The compound of the formula (V) is 3,5-bis(bromomethyl)toluene, 3,5-bis(chloro methyl)toluene, 3,5-bis(iodomethyl)toluene or preferably is 3,5-bis(bromo methyl)toluene.

The metal cyanide used in step (b) of cyanation of the compound of the formula (V) is selected from sodium cyanide, potassium cyanide, cuprous cyanide, lithium cyanide or trimethylsilyl cyanide or preferably is sodium cyanide. The organic solvent used in step (b) of cyanation of the compound of the formula (V) is selected from halogenated solvent such as dichloromethane, chloroform or aliphatic or aromatic hydrocarbon or preferably is dichloromethane. The catalyst used in step (b) of cyanation of the compound of the formula (V) is selected from potassium iodide, sodium iodide or ammonium iodide or a phase transfer catalyst selected from crown ethers, quaternary ammonium salt or phosphonium salts or preferably is quaternary ammonium salt. Preferably the cyanation of step (c) is carried out at 50° C.

The base used in step (c) of methylation of the compound of the formula (VI) is selected from alkali metal hydroxides, alkaline-metal hydroxide, metal oxide or organic base or preferably is selected from n-butyl lithium, sodium tertiary butoxide, potassium tertiary butoxide, sodium hydride or more preferably is sodium hydride. The organic solvent used in step (c) of methylation of the compound of the formula (VI) is selected from aliphatic or aromatic hydrocarbons like toluene or xylene; dimethyl sulphoxide; ether such as diphenyl ether; dimethyl acetamide; dimethyl formamide or 1,2-dimethoxy/diethoxy ethane or preferably is dimethylformamide. The methylation of step (c) is preferably carried out at 5 to 15° C.

The N-halosuccinimide used in the halogenation step (d) of halogenation of the compound of the formula (II) is N-bromosuccinimide or N-chlorosuccinimide or N-iodosuccinimide or preferably is N-bromosuccinimide. The compound of the formula (M) is 2,2′-(5-bromomethyl-1,3-phenylene)di(2-methyl propionitrile), 2,2′-(5-chloromethyl-1,3-phenylene)di(2-methyl propionitrile) or 2,2′-(5-iodomethyl-1,3-phenylene)di(2-methylpropionitrile) or preferably is 2,2′-(5-bromomethyl-1,3-phenylene)di(2-methylpropionitrile). The catalyst used in step (d) is selected from dibenzoyl peroxide or azobis isobutyronitrile. Preferably the chlorinated solvent used in step (d) is 1,1,2-ethylene trichloride. Preferably the halogenation of step (d) is carried out at 88 to 92° C.

Preferably the treatment of step (e) is carried out at 45 to 50° C.

The purification of step (f) by column chromatography is carried out using the stationary phase selected from silica gel or alumina and the mobile phase selected from aliphatic or aromatic hydrocarbons, alcoholic solvent, ketonic solvent, aliphatic esters, chlorinated solvent or mixtures thereof or preferably is ethyl acetate and hexane. The purification of step (f) by recrystallization is carried out using the solvent selected from toluene, xylene, methanol, ethanol, isopropanol, acetone, methyl ethyl ketone, methyl isobutyl ketone, ethyl acetate, iso-propyl acetate, petroleum ethers, hexane or water or mixture thereof or preferably is mixture of ethyl acetate and hexane or acetone and water.

According to the invention 2,2′-[5-(1H-1,2,4-triazole-1-ylmethyl)-1,3-phenylene]di(2-methylpropionitrile) of the formula (I) is obtained in high yield (about 34%) and in high purity (about or greater than 99.5%). It also avoids the use of solvents, which are harmful and hazardous to the environment like carbon tetrachloride. It does not employ purification of intermediate compounds by flash column chromatography and crystallization at each step but still gave highly pure Anastrozole in high yield. It reduces the volume of the solvents employed and the process time. For the above reasons, the process of the invention is cost effective and economical, efficient, environmental friendly, simple and easy to carry out and is also of reduced duration. The invention also provides starting material of the formula (V) and the process for the preparation thereof from mesitylene.

The process of the invention is illustrated by the following example, which is by way of illustration only and not to be considered to limit the scope of the invention in any manner.

### EXAMPLE

### a) Preparation of 3,5-Bis(Bromomethyl)toluene (V; Wherein X═Br)

Mesitylene (10 gm, 0.083 moles) was refluxed with N-bromosuccinimide (31.5 gm, 0.176 moles) in 1,1,2-ethylenetrichloride (100 ml) under the influence of light (tungsten filament lamp 300-600 watts) for 2-4 hour. The reaction mixture was cooled gradually to room temperature and filtered. The mother liquor was concentrated under vacuum to give yellowish oily residue. The residue obtained was crystallized from a mixture of isopropanol and toluene in a ratio of 9:1 to give 3,5-bis(bromomethyl)toluene (V). [Yield: 65%, m.p. 65-66° C.]

### b) Preparation of 2,2′-(5-Methyl-1,3-phenylene)diacetonitrile (VI)

A mixture of 3,5-bis(bromomethyl)toluene(V) (10 gm, 0.035 moles), sodium cyanide (9.3 gm, 0.189 moles), tetrabutylammoniumbromide (0.266 gm) was heated in dichloromethane (40 ml) and water (16 ml) at 50° C. for 8 hours. The reaction mixture was cooled and diluted with water (30 ml). The organic layer was separated and washed with water (2×20 ml) and further dried over anhydrous sodium sulphate. The organic layer was concentrated under vacuum to give a residue. This residue obtained was crystallized from isopropanol to give solid 2,2′-(5-methyl-1,3-phenylene)diacetonitrile (VI). [Yield: 81.9%, m.p. 75-78° C.]

### c) Preparation of 2,2′-(5-Methyl-1,3-phenylene)di(2-methylpropiononitrile) (II)

A solution of 2,2′-(5-methyl-1,3-phenylene)diacetonitrile (VI) (5 gm, 0.029 moles) in N,N-dimethylformamide (100 ml) was stirred under nitrogen. To this, methyl iodide (18.1 gm, 0.127 moles) was added in a one lot at 0-5° C. 60% sodium hydride (5.29 gm, 0.22 moles) was added in a small portion at a time at 0-10° C. to the reaction mixture for 1.5 hour. The reaction mixture was stirred for 30 min at 10-15° C. and further stirred for 4 hour. The reaction mixture was poured into the ice-water and solid obtained was filtered. The filtrate was extracted with ethyl acetate. The solid was dissolved in the ethyl acetate extract and washed twice with water. The ethyl acetate layer was separated and concentrated to give a residue. The residue obtained was crystallized from ethyl acetate-hexane (20 ml: 10 ml) to give a solid 2,2′-(5-methyl-1,3-phenylene)di(2-methylpropiononitrile) (II). [Yield: 67%, m.p. 126-127° C.]

### d) Preparation of 2,2′-(5-Bromomethyl-1,3-phenylene)di(2-methyl propionitrile) (III; wherein X═Br)

A mixture of 2,2-(5-methyl-1,3-phenylene)di(2-methylpropionitrile) (II) (4 gm, 0.0176 moles), N-bromosuccinimide (3.3 gm, 0.018 moles) and benzoyl peroxide (0.4 gm) in 1,1,2-ethylenetrichloride (40 ml) was refluxed for 2-3 hour. Reaction mixture was cooled gradually to room temperature and filtered. The mother liquor was concentrated under vacuum to give yellowish oily residue. The residue obtained was crystallized from hexane to give a solid 2,2′-(5-bromomethyl-1,3-phenylene)di(2-methylpropionitrile) (IIIb), which was used without further purification for next step. [Yield: 74%]

### e) Preparation of 2,2′-[5-(1H-1,2,4-triazole-1-yl-methyl)-1,3-phenylene]di(2-methyl propionitrile) (1)

A mixture of 2,2′-(5-bromomethyl-1,3-phenylene)di(2-methylpropionitrile) (V) (4 gm, 0.013 moles) and 1,2,4-triazole sodium salt (2.40 gm, 0.026 moles) in N,N-dimethylformamide (28 ml) was heated under stirring at 45-50° C. for 4 hour. The reaction mixture was cooled, poured into the ice-water and extracted with ethyl acetate. The ethyl acetate layer was separated and concentrated to give a residue. This residue was diluted with water, acidified with concentrated hydrochloric acid (adjusted the pH tol-2). The reaction mixture was heated at 50° C. and subsequently filtered the hot solution. The mother liquor was treated with ammonia solution at 0-5° C. (adjusted the pH to 8-9) and extracted with ethyl acetate. The ethyl acetate layer was separated and concentrated to obtain a residue (3.0 g).

### f) Purification of Anastrozole by Column Chromatography and Crystallization

This residue was purified through column chromatography by using silica gel column and ethylacetate-hexane (740 ml: 950 ml) as eluent. The solid (2.0 g) obtained was recrystallized using a mixture of acetone-water (12 ml: 20 ml) or ethylacetate-hexane mixture (9 ml to 18 ml) to obtain 2,2′-[5-(1H-1,2,4-triazole-1-ylmethyl)-1,3-phenylene]di(2-methyl propionitrile) (1). [Yield: 34.0%, purity, 99.9%].

1. A process for the preparation of 2,2′-[5-(5-1H-1,2,4-triazole-1-ylmethyl)-1,3-phenylene[di(2-methylpropionitrile) of the formula (I) 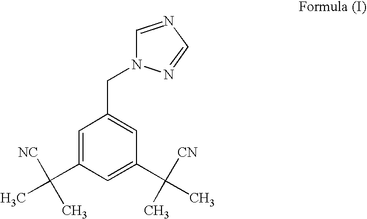
comprising the following steps;
a. preparing 3,5-bis(halonethyl)toluene of the formula (V) 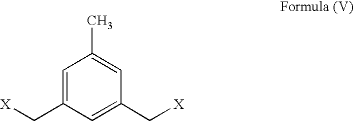
wherein X is Cl (a); Br (b) or I (c)
by reacting mesitylene with N-halosuccinimidc in a 1,1,2-ethylenetrichloride as a solvent in the presence of a catalyst selected from light or dibenzoyl peroxide or azobis isobutyronitrile at temperature of 60 to 100° C. followed by recrystallization of the compound of the formula (v) using a solvent selected from alcohols, ketones, others, esters or aliphatic or aromatic hydrocarbons or mixtures thereof;
b. preparing 2,2′-(5-methyl-1,3-phenylene)diacetonittile of the formula (VI) 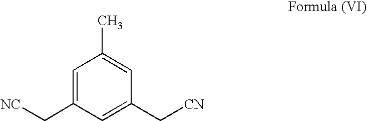
by cyanation of the compound of the formula (V) with metal cyanide in water or organic solvent or mixture thereof in the presence of a catalyst at a temperature of 40 to 60° C.;
c. preparing 2,2′-(5-methyl-1,3-phenylene)di(2-methyl-propiononitrile) of the formula (II) 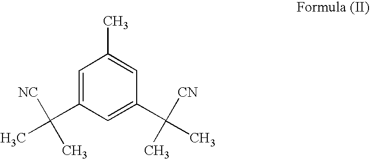
by methylating the compound of the formula (VI) with iodomethane in the presence of base and an organic solvent at temperature of 0 to 15° C.;
d. preparing 2,2′-(5-halomethyl-1,3-phenylene)di(2-methyl propionitrile) of the formula (III) 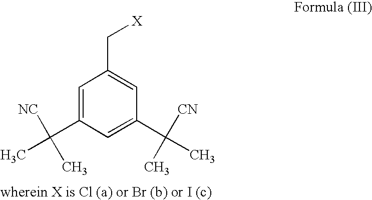
by halogenating the compound of the formula (II) with N-halosuccinimide in 1,1,2-ethylene trichloride as a solvent in the presence of a catalyst such as dibenzoyl peroxide or azobis isobutyronitrile at temperature of 60 to 100° C.;
e preparing 2,2′-[5-(1H-1,2,4-triazole-1-ylmethyl)-1,3-phenylene]di(2-methyl propionitrile) of the formula (I) by treating the compound of the formula (III) with potassium or sodium salt of 1,2,4-triazole in dimethyl formamide at temperature of 20 to 50° C.; and
f. purifying the compound of the formula (I) by column chromatography using a stationary phase and a mobile phase followed by recrystallization with a solvent or mixture of solvents to obtain highly pure compound of the formula (I).

2. The process as claimed in claim 1, wherein the N-halosuccinimide used in the reaction of mesitylene of step (a) is N-bromosuccinimide.

3. The process as claimed in claim 1, wherein the catalyst used in the reaction of mesitylene of step (a) is light.

4. The process as claimed in claim 1, wherein the reaction of mesitylene of step (a) is carried out at 88 to 92° C.

5. The process as claimed in claim 1, wherein the compound of formula (V) is recrystallized using the mixture of toluene and isopropanol.

6. The process as claimed in claim 1, wherein the metal cyanide used in step (b) of cyanation of the compound of the formula (V) is sodium cyanide.

7. The process as claimed in claim 1, wherein the organic solvent used in step (b) of cyanation of the compound of the formula (V) is dichloromethane.

8. The process as claimed in claim 1, wherein the catalyst used in step (b) of cyanation of the compound or the formula (V) is quaternary ammonium salt.

9. The process as claimed in claim 1, wherein the cyanation of step (b) is carried out at 50° C.

10. The process as claimed in claim 1, wherein the base used in step (c) of methylation of the compound of the formula (VI) is sodium hydride.

11. The process as claimed in claim 1, wherein the organic solvent used in step (c) of methylation of the compound of the formula (VI) is dimethyl formamide.

12. The process as claimed in claim 1, wherein the methylation of step (c) is preferably carried out at 5 to 15° C.

13. The process as claimed in claim 1, wherein the N-halosuccinimide used in the step (d) halogenation is N-bromosuccinimide.

14. The process as claimed in claim 1, wherein the halogenation of step (d) is carried out an 88 to 92° C.

15. The process as claimed in claim 1, wherein the treatment of step (e) is carried out at 45 to 50° C.,

16. The process as claimed in claim 1, wherein the purification of step (f) by column chromatography is carried out using the stationary phase selected from silica gel or alumina and the mobile phase is ethyl acetate and hexane.

17. The process as claimed in claim 1, wherein the purification of step (f) by recrystallization is carried out using the solvent selected from mixture of ethyl acetate and hexane or acetone and water.

18. A process for the preparation of 3,5-bis(halomethyl)toluene of the formula (V) 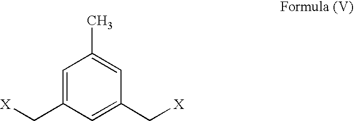
wherein X is Cl (a); Br (b) or T (c)
comprising reacting mesitylene with N-halosuccinimide in 1,1,2-ethylenetrichloride as a solvent in the presence of a catalyst selected from light or dibenzoyl peroxide or azobis isobutyronitrile at a temperature of 60 to 100° C. followed by recrystallization of the compound of the formula (v) using a solvent selected from alcohols, ketones, ethers, esters or aliphatic or aromatic hydrocarbons or mixtures thereof.

19. The process as claimed in claim 18, wherein the N-halosuccinimide used in the reaction of mesitylene is N-bromosuccinimide.

20. The process as claimed in claim 18, wherein the catalyst used in the reaction of mesitylene is light,

21. The process as claimed in claim 18, wherein the reaction of mesitylene is carried out at 88 to 92° C.

22. The process as claimed in claim 18, wherein the compound of formula (V) is recrystallized using the mixture of toluene and isopropanol.
